(19) United States
(12) Patent Application Publication
(10) Pub. No.: US 2014/0162215 A1
(43) Pub. Date: Jun. 12, 2014

(54) ABUTMENT FOR AN ARTIFICIAL DENTAL PROSTHESIS, ARTIFICIAL DENTAL PROSTHESIS AND A METHOD FOR PRODUCING AND/OR IMPLANTING AN ARTIFICIAL DENTAL PROSTHESIS

(71) Applicant: Johan Feith, Eurasburg (DE)

(72) Inventor: Johan Feith, Eurasburg (DE)

(73) Assignee: ZV3-ZIRCON VISION GMBH, Wolfratshausen (DE)

(21) Appl. No.: 14/233,583

(22) PCT Filed: Jul. 17, 2012
(86) PCT No.: PCT/EP2012/063939

§ 371 Date: Jan. 17, 2014

(30) Foreign Application Priority Data
Jul. 19, 2011 (DE) 102011051930.0
Aug. 12, 2011 (DE) 102011052644.7

(51) Int. Cl. A61C 8/00; A61C 13/00; A61K 6/02

(52) U.S. Cl. CPC A61C 8/006; A61C 8/0089; A61C 8/0016; A61K 6/0205; A61C 8/0069; A61C 8/0022; A61C 13/0006
USPC 433/174; 433/173; 433/201.1

(57) ABSTRACT

An abutment is provided which enables an associated anchoring part to be introduced into a bone without the anchoring part being damaged. A corresponding abutment includes: an abutment upper part disposed along a longitudinal axis and an abutment lower part. For form-fitting insertion of the abutment into an abutment receiving region of an anchoring part, the abutment lower part has a profile such that a torque applied to the abutment is transferable to the anchoring part. The abutment upper part includes a tool receiving portion for form-fittingly receiving a tool.

### CROSS-REFERENCE TO RELATED APPLICATIONS

This application is a Section 371 of International Application No. PCT/EP2012/063939, filed Jul. 17, 2012, which was published in the German language on Jan. 24, 2013, under International Publication No. WO 2013/011003 A1 and the disclosure of which is incorporated herein by reference.

### BACKGROUND OF THE INVENTION

The present invention relates to an abutment for an artificial dental prosthesis, an artificial dental prosthesis having a corresponding abutment and a method for producing and/or implanting an artificial dental prosthesis.

Two-part dental implants are known from the prior art (U.S. Patent Application Publication No. 2003/0104338 A1). They are made up of an anchoring part and an abutment. Following implantation of the anchoring part and having waited for any potentially necessary healing time to pass, the abutment is screwed or bonded onto the anchoring part. The abutment then supports the crown or the appropriate superstructure. A gap, in which bacteria can become established, exists between the anchoring part and the abutment. This can sometimes lead to bacterially-induced bone resorption. The profile of the natural gingival boundary also changes because the bone recedes. The gingiva recede or become displaced in such a way that transitions between the implant and the crown sometimes become visible. This disrupts the visual appearance of the artificial dental prosthesis. Titanium implants which become exposed are particularly unappealing and aesthetically undesirable.

German published patent application DE 101 59 683 A1 thus proposes the use of one-piece implants, particularly based on zirconium oxide, the abutment and the anchoring part being made in one piece. After sintering, such one-piece implants are practically ungrindable using conventional means. This means that zirconium oxide can be ground even in the sintered state, but microcracks occur in the process and as a result the artificial dental prosthesis occasionally becomes unusable. Moreover, when grinding zirconium oxide, the material is heated in such a manner that cells adjacent to the implant die off. It is therefore impossible to make subsequent adjustments to the implant. The requirements on the implant production process and on the person placing the implant are correspondingly high.

U.S. provisional patent application 60/438,266 proposes a two-part implant, comprising an anchoring part and an abutment made of titanium. The abutment is partially inserted into the anchoring part and joined thereto by way of a screw thread. The implant has an external contact joint which is disposed at a distinct distance in the bone when the implant is placed. The implant is designed such that the external contact joint is sealed at least in sections by a crown to be attached.

Titanium implants should also not be ground in situ. As such implants also have a high level of heat conductivity, the local temperature rise due to grinding is distributed through the whole implant. The implant heats up and bone cells which are in direct contact with the implant die off. Any healing success already achieved is reversed. Moreover, metal splinters, which are detached and greatly accelerated by the grinding tool, can penetrate the patient's gums. It is frequently impossible to remove these splinters subsequently. They remain in the gum and sometimes noticeably discolor the tissue.

Allowing for these deficiencies, European patent application publication No. EP 2 146 665 A1 (WO 2008/128620) proposes the use of a three-part dental implant comprising an anchoring part, an abutment and a crown. The abutment should preferably be made of plastic so that it can easily be ground. The anchoring part should be made at least in sections of a technical ceramic which is significantly harder than the plastic of the abutment. When using technical ceramics, the problem arising is that they must be introduced into the bone with great care. If an appropriate anchoring part is damaged while it is being introduced (e.g., a portion breaks off), it is extremely problematic to remove it. For example, such an anchoring part is very difficult to dissect with the result that significant loss of tissue and bone may be expected.

In addition, a metal tool is usually used to ensure introduction of the anchoring part and during this procedure abrasion can occur such that deposits are left behind. Such deposits can significantly disrupt the creation of a bonded joint.

### BRIEF SUMMARY OF THE INVENTION

Based upon this prior art, in particular based upon EP 2 146 665, it is an object of the present invention to make the process of introducing the anchoring part easier. In particular, this introduction process should be made safer and more efficient.

In particular, the object is achieved by an abutment for an artificial dental prosthesis which comprises:
an abutment upper part disposed along a longitudinal axis and
an abutment lower part disposed along the longitudinal axis, which for form-fitting insertion of the abutment into an abutment receiving region of an anchoring part, has a profile such that a torque applied to the abutment is transferable to the anchoring part, while the abutment upper part comprises a tool receiving portion for form-fittingly receiving a tool.

Tools which are used to introduce the anchoring part should therefore no longer attach directly to the anchoring part but to a tool receiving portion provided for this purpose on the abutment, in particular the abutment upper part. The forces applied (approx. 20 to 50 Nm, in particular 30 to 40 Nm) are then transferred indirectly to the anchoring part. In this respect, for example, if the tool slips off it results in damage to the abutment—not in damage to the anchoring part. It is considerably easier to replace the abutment than the anchoring part so as to avoid injuring the patient. The abutment can be an abutment that later forms part of the artificial dental prosthesis or, alternatively, an abutment that is removed after introduction of the anchoring part and is preferably replaced with a different abutment. In the present application, an abutment can be any 3-dimensional body which is suitable for having an anchoring part placed preferably form-fittingly onto it. A corresponding abutment can only be used as an introduction aid for introducing the anchoring part into the biological tissue. It is possible but not absolutely essential for the abutment to form a functional element of the complete dental prosthesis, comprising crown and anchoring part.

The tool receiving portion can comprise an appendage which sits on the abutment upper part along the longitudinal axis, in particular on the side directed away from the abutment. The tool receiving portion thus protrudes such that it can be removed after introducing the anchoring part, e.g. it can be ground or cut off

The abutment can have a predetermined breaking point which limits torque transmission between abutment upper part and abutment lower part and/or between tool receiving portion and abutment lower part. The abutment can also have a limitation of torque transmission which prevents too great a force being applied on the anchoring part. This force limitation can be guaranteed by a predetermined breaking point which results in a controlled fracture of the abutment if forces that are too high are applied. This can effectively prevent damage to the anchoring part.

The abutment can comprise at least one notch for providing the predetermined breaking point.

The profile of the abutment can comprise a polygonal section and/or a hexalobular shape in order to join the abutment form-fittingly to the anchoring part.

The tool receiving portion can comprise a polygonal section and/or a hexalobular shape and/or a receiving portion for polygonal sections and/or a receiving portion for a hexalobular shape. Theoretically, it would be conceivable to design the tool receiving portion similarly to a slotted or cross head screw. However, hexalobular shapes or polygonal profiles are to be preferred. For example, profiles that are known from internal torx and external torx screws can be used. Alternatively, polygonal profiles (e.g. hexagonal recess) can be used. Such profiles are particularly suitable for transmitting high torques without causing any damage to the corresponding profiles—that is to say the tool receiving portion and thus the abutment. Moreover, such shapes provide better guidance of the applied tool. Depending on use, a propeller shape (bipartite and/or tripartite) can be selected. The cross-section can have the shape of a figure eight.

The abutment can be designed to be grindable at least in sections. Preferably, it comprises plastic, in particular fiberglass-reinforced and/or carbon fiber-reinforced plastic. Such a design enables the abutment to be adapted to individual circumstances, e.g. by shortening, milling the circumference, creating an inclination of the abutment. Theoretically, it would be conceivable to grind in situ. Plastics are thermal insulators with the result that even grinding within the oral cavity does not result in heating of the anchoring part. The fiberglass or carbon fiber reinforcement results in a very stable abutment. Nevertheless, plastic is so flexible that any excessive forces which occur are not passed on directly to the anchoring part.

If a fiberglass and/or carbon fiber reinforcement is provided, the fibers can preferably be aligned substantially along the longitudinal axis of the abutment. Experiments have shown that this produces an extremely stable abutment which can transfer rotational forces that are greater than 30 Nm. The abutment is preferably produced by the pultrusion process. Grinding can take place thereafter.

The abutment lower part and/or the abutment receiving region may taper along the longitudinal axis (preferably towards the bottom).

The abutment upper part may be widened in relation to the abutment lower part and/or be configured as outwardly protruding to create a contact surface, the contact surface preferably extending essentially perpendicular to the longitudinal axis. The transition between vertical surfaces and horizontal surfaces may be designed as right-angled, acute-angled or stepped. For example, the abutment may have a mushroom-shaped design overall. The contact surface may be used to create a contact closure with a corresponding surface on the anchoring part. In this respect, it is possible to create a bonded joint between the abutment and the anchoring part.

The abutment part/the introduction aid may comprise a solid basic body of plastic, in particular fiberglass-reinforced and/or carbon fiber-reinforced plastic. A solid body is particularly suitable for transferring the forces applied (20-35 Nm for a technical ceramic, 20-50 Nm for a metal). The fiberglass-reinforced plastic may be a fiber/plastic composite on an epoxy resin base. In one embodiment, the proportion of fiber in the fiberglass-reinforced and/or carbon fiber-reinforced plastic may be greater than 50% and/or greater than 60% and/or greater than 70%. It has emerged that a particularly high proportion of fiber leads to a discoloration of the material, which is easy to observe visually if the material is over-tightened—that is to say, if too much force is applied. The dentist may see this as a signal that the abutment is unusable. Moreover, the high proportion of fiber means that a relatively defined resistance can be established even with a solid design of the abutment. In this respect, the abutment can be dimensioned in such a way that material fatigue occurs at a predefined force (e.g., at 35, 40 or 45 Nm). In this respect, the transmission of force beyond these limits is effectively prevented.

Moreover, the abutment can be designed in such a way that it can be inserted form-fittingly into the anchoring part designed for this purpose.

Moreover, the object referred to above is achieved by an artificial dental prosthesis with an implant for receiving a crown, the implant having an abutment like the one described previously and an anchoring part. The anchoring part may comprise an abutment receiving region for receiving the abutment and may be formed at least in sections from a first material, the first material belonging preferably to the material group of technical ceramics, in particular oxide ceramics. Similar advantages emerge, such as have already been described in connection with the abutment.

The anchoring part may have a shoulder section or a frustoconical section, in particular with a concave circumferential surface for receiving part of a crown. A crown receiving region can be created due to the special design. This is especially suitable for forming a preparation margin, such that the crown can be applied to the anchoring part and optionally the abutment. The crown receiving region ensures that no cavities or projections which support bacterial attack arise during this process of applying material.

The anchoring part may comprise at least one threaded section for screwing the anchoring part into a bone. Thus, the anchoring part can preferably by anchored in the bone by way of a thread. Introduction of the anchoring part is made easier in that threaded sections are provided which enable the anchoring part to be screwed in like a screw. The design of the abutment with the tool receiving portion according to the invention becomes particularly important at this point as the torque can be used to screw the anchoring part into the bone.

The anchoring part may be formed in such a way that at least one cross-sectional area has an essentially oval, in particular elliptical, area boundary. This cross-sectional area emerges preferably if a cut is made through the anchoring part perpendicular to its longitudinal axis. Depending on which tooth the artificial dental prosthesis is intended to replace, it is desirable to provide anchoring parts of different design. For example, when replacing a premolar, very little space remains between the adjacent teeth for the anchoring part. Therefore, the anchoring part must be very small, for example with a diameter of the subgingival section less than 5 mm, in particular less than 4.5 mm. In order to model and/or place the crown in an appropriate manner, widening is carried out in the upper region (e.g. isogingivally and/or in the shoulder section). To allow here for natural circumstances, this widening can have an essentially oval design in a plan view. The anchoring part is preferably designed in such a way that, particularly in the upper region, a cross-sectional area emerges which is axisymmetrical to at least one axis of symmetry that extends from the palatal side of the artificial dental prosthesis to its labial side in the inserted state.

It is advantageous particularly with such small anchoring parts if the abutment receiving region comprises an elongated slot, in particular an elongated hole. This elongated hole may extend along the axis of symmetry. It is therefore possible with a very small anchoring part to transfer relatively high torsional forces during introduction. Moreover, this design has the advantage that it is possible to introduce forces which act on the crown into the anchoring part in an appropriate manner. A high level of stiffness emerges particularly along the axis of symmetry such that the usual forces can be dissipated in an optimum manner.

If the anchoring part comprises a threaded section, such as has already been described, the thread should be equipped with a relatively small thread pitch. The thread is preferably designed in such a manner that the resulting difference in height is less than 2 mm, in particular less than 1 mm, per turn. In this respect it is possible to align the anchoring part advantageously (e.g., because of a predefined alignment of the anchoring part and/or the abutment receiving region). In this respect it is possible to guarantee a perfect fit of the anchoring part, where a 180° turn during insertion results, for example, in only a slight difference in height.

Moreover, the object referred to is achieved by a method for producing and/or implanting an artificial dental prosthesis. This is preferably an artificial dental prosthesis such as has already been described. The method comprises the following steps:
Production of a preferably individualised anchoring part with an abutment receiving region;
Introduction of a section of an abutment into the abutment receiving region for force-fittingly, in particular form-fittingly, joining the abutment to the anchoring part;
Attachment of a tool, preferably to the abutment;
Application of a torque by means of the tool in order to screw the anchoring part into a bone.

Here too, similar advantages emerge, such as have already been described in connection with the device.

The method can additionally comprise a removal of sections of the abutment for adapting the abutment to patient-specific circumstances. Such a removal can take place in situ or in the laboratory or workshop. To do this, it is necessary to adapt the abutment to the patient's individual circumstances (e.g., alignment of the teeth, height of the teeth, shape of the gums). The abutment is preferably supplied as a standard part although it has a certain excess length so that any shapes can be milled out of the abutment, in particular out of the abutment upper part. The abutment then serves as a type of backbone for the artificial dental prosthesis. In particular, the force fit is improved between a crown to be attached and the anchoring part.

The method may additionally comprise the following steps:
Production of a zirconium oxide green compact for producing an anchoring part;
Firing/sintering of the green compact;
Shading of at least one colored section of the green compact prior to the step of firing/sintering of the green compact.

Moreover, the method may include roughening, in particular blasting, of at least one special section of the green compact prior to sintering or firing of the green compact.

Roughening may be blasting with aluminum oxide.

The method according to the invention may additionally comprise the step of taking out the introduced abutment and inserting a new abutment for use in conjunction with a crown. In this respect, it is conceivable to use the first-mentioned abutment merely as an introduction aid and to replace it after introducing the anchoring part. The second abutment may then form a fixed component of the artificial dental prosthesis. The first and the second abutment preferably create a positive fit with the anchoring part while using the abutment receiving region.

Further advantageous embodiments emerge from the dependent claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. The figures show:

FIG. 1 is a sectional view through an artificial dental prosthesis according to an embodiment of the invention with a crown, an anchoring part and an abutment;

FIG. 2 is a plan view of the anchoring part from FIG. 1;

FIG. 3 is perspective view of a first embodiment of an abutment;

FIG. 4 is a perspective view of a second embodiment of an abutment having a tool receiving portion in the shape of a clover leaf;

FIG. 5 is a perspective view of a third embodiment of an abutment having a triangle as the tool receiving portion;

FIG. 6 is a perspective view of a fourth embodiment of an abutment having a triangle as the tool receiving portion;

FIG. 7 is a perspective view of a fifth embodiment of an abutment (square);

FIG. 8 is a perspective view of a sixth embodiment of an abutment (conical);

FIG. 9 is a sectional view of the inserted anchoring part from FIG. 1;

FIG. 10 is a plan view of a further anchoring part;

FIG. 11 is a perspective view of a seventh embodiment of an abutment (elliptical); and

FIG. 12 is a sectional view of the inserted anchoring part from FIG. 10.

### DETAILED DESCRIPTION OF THE INVENTION

In the following description, the same reference numerals are used for identical parts and parts acting in an identical manner.

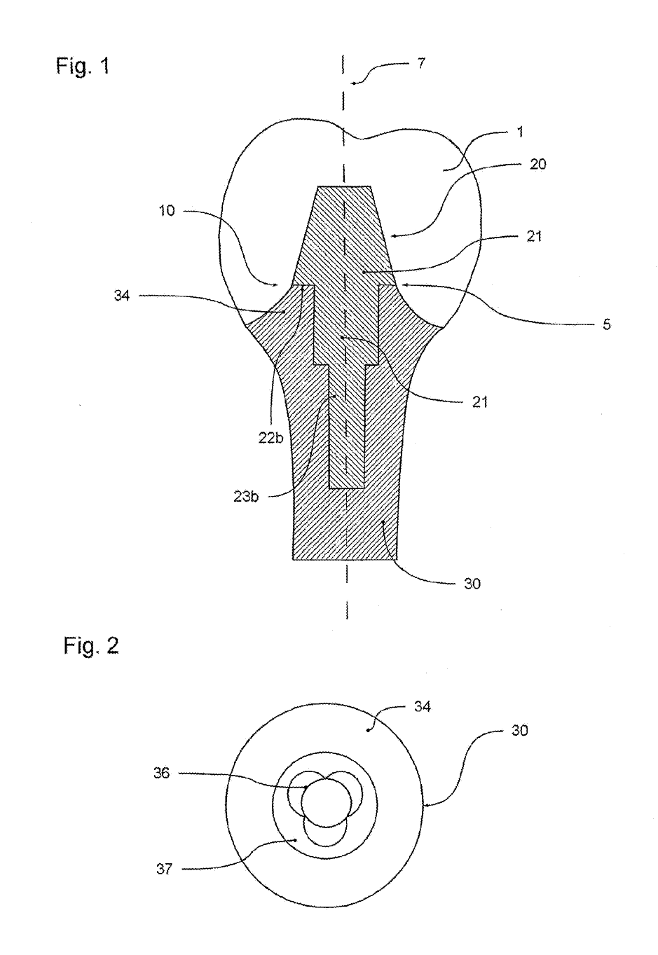

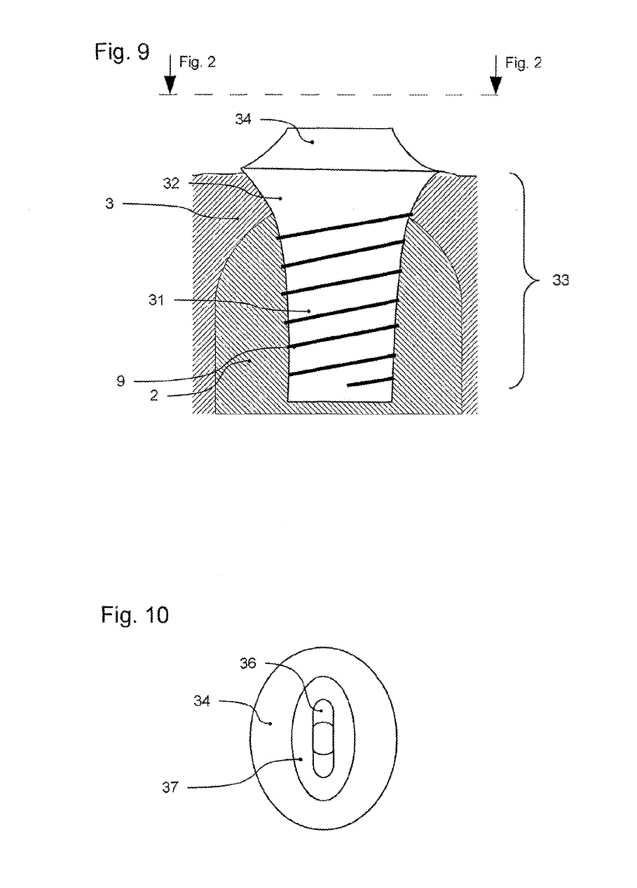

An artificial dental prosthesis according to the invention comprises, as can be seen from FIG. 1, a crown 1, an abutment 20 and an anchoring part 30, whereby the abutment 20 and the anchoring part 30 form a two-part implant 10 on which the crown 1 rests. As can be seen from FIGS. 2 and 8, the anchoring part 30 in the embodiment described is an essentially rotationally symmetrical pin which extends along a longitudinal axis 7 and is inserted into the gum 3 and the bone 2. In a further embodiment, the anchoring part 30 may be executed asymmetrically and adapted to individual circumstances. According to the lateral view (cf. the schematic illustration from FIG. 9), the diameter of the anchoring part 30 increases from the bottom upwards in a subgingival section 33 and decreases again beyond the subgingival section 33.

This section beyond the subgingival section 33 is referred to as a shoulder section 34 which ends in a plateau section that is referred to as the receiving region 37. The anchoring part 30 is preferably individualized specific to the patient in such a way that the transition between subgingival section 33 and shoulder section 34 runs isogingivally.

The frustoconical shoulder section 34 has a concave circumferential surface which receives crown 1.

As can be seen from FIG. 1, a partial section of the abutment 20 rests on the plateau section or receiving region 37. The receiving region 37 can be bonded to the abutment 20 on placing the implant.

A receiving channel 36 which also receives a section of the abutment 20 extends along the longitudinal axis 7 inside the anchoring part 30.

In a first embodiment, the upper section of the receiving channel 36 is designed as a hexalobular shape and the lower section as a cylinder. Ultimately, in cross-section the upper section of the receiving channel 36 has a design that corresponds to three overlapping circles—similar to a clover leaf. The corresponding cross-section through the lower section of the receiving channel 36 is circular.

The abutment 20 comprises an abutment lower part 23 corresponding in design to the receiving channel 36, on which abutment lower part an abutment upper part 21 is mounted. The abutment lower part 23 is divided into a drive part 23a (corresponding to the upper section of the receiving channel 36) and a retention part 23b (corresponding to the lower section of the receiving channel 36). In cross-section, the drive part 23a of the abutment lower part 23 protrudes relative to the retention part 23b, the abutment upper part 21 overhanging it. In the fully implanted state, the frustonconically designed abutment upper part 21 aligns with the shoulder section 34, in particular with the concave circumferential surface, of the anchoring part 30 and rests by an abutment upper part base area 22b on the receiving region 37 of the anchoring part 30. The integrally constructed abutment 20 with the anchoring part 30 forms a contact joint 5 which is covered and sealed by the crown 1.

In a preferred embodiment, the anchoring part 30 is designed as a technical ceramic, and the abutment 20 is constructed of plastic. A technical ceramic, for example zirconium oxide, can also be used to produce the crown 1. In this respect, the artificial dental prosthesis has a flexible “backbone” in the shape of the abutment 20 and a rigid shell in the shape of the crown 1 and the anchoring part 30.

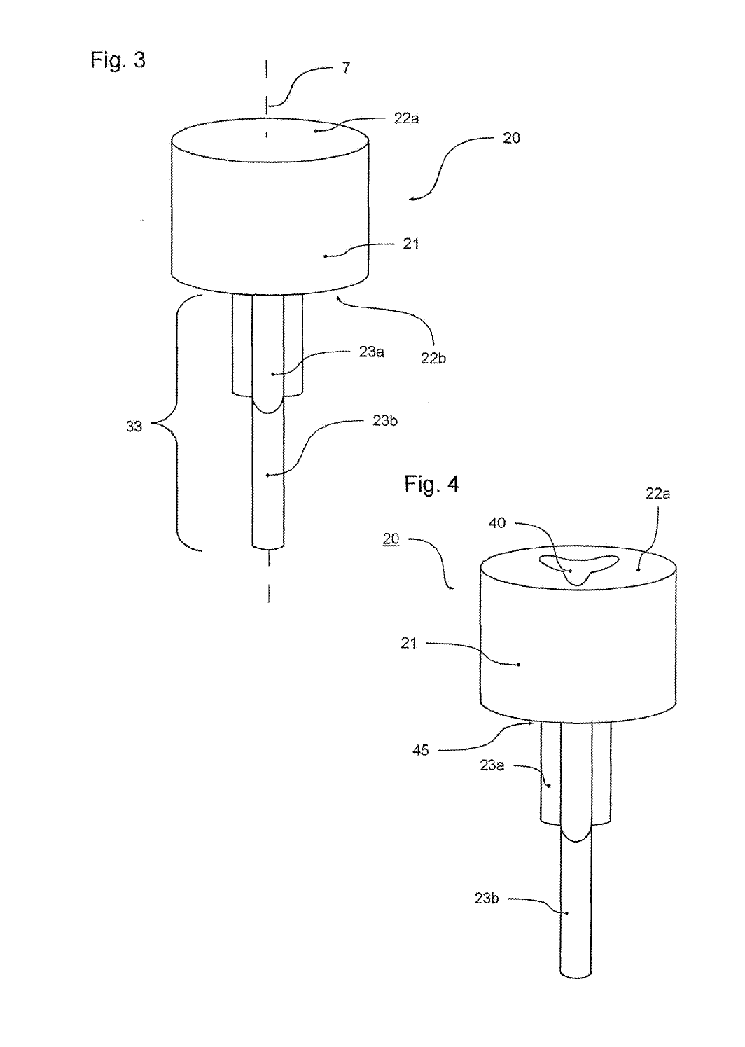

The abutment can preferably by adapted to patient-specific circumstances. Based on FIG. 3, it is explained how an appropriately individualized abutment 20 can be produced. In an initial shape, the abutment 20 has, as already explained, an abutment upper part 21 and an abutment lower part 23 with a drive part 23a and a retention part 23b. The abutment upper part 21 is executed as a cylinder and is dimensioned such that it overhangs the receiving region 37 laterally. The cylinder has an abutment upper part cover area 22a and the abutment upper part base area 22b. After implantation of the anchoring part 30, the abutment 20, with the drive part 23a formed correspondingly to the receiving channel 36 and the retention part 23b, is inserted into the receiving channel 36. The abutment upper part base area 22b and the receiving region 37 make contact with each other in the inserted state of the abutment 20. After insertion, the abutment upper part 21 can be adapted in situ or in a model in such a way that patient-specific circumstances are taken into consideration. For example, the abutment 20, in particular the abutment upper part 21, can be ground in such a manner that a frustoconical shape emerges, such as is shown in FIG. 1.

In an alternative embodiment, pre-assembled abutments 20 can be provided.

An essential part of the present application deals with the effective insertion of the anchoring part 30 in the bone 2 and the gum 3. For this, the anchoring part 30, as shown in FIG. 9, comprises a threaded section 31 which enables the anchoring part 30 to be screwed into the bone 2. It is possible to provide tools which engage in the receiving channel 36 of the anchoring part 30 and make screwing in easier. During this, however, the anchoring part 30 can easily be damaged. Moreover, such high forces may be applied that the patient's bone 2 is damaged.

The present invention solves this problem in that prior to screwing the anchoring part 30 into the bone 2, an abutment is used, such as shown in FIG. 4, for example. Due to the form-fit of the retention part 23b with the receiving channel 36, a force-locked joint is thus created between the abutment 20 and the anchoring part 30. The force-fit is created in particular in respect of a rotational movement about the longitudinal axis 7. According to the invention, a tool receiving portion 40 is provided on the abutment 20, which in particular enables form-fitting application of the tool. In the embodiment shown in FIG. 4, the tool receiving portion 40 is a recess provided on the abutment upper part cover area 22a, which ultimately has a similar design to the upper region of the receiving channel 36. The recess is thus designed in the form of a hexalobular shape, which looks similar to three overlapping holes or a cloverleaf. The dentist working here can therefore use the same instrument that was originally used to directly screw in the anchoring part 30, in order to screw in the anchoring part 30 using the abutment 20 according to the invention.

In one embodiment, the abutment 20 according to the invention has a predetermined breaking point 45 which is located between the abutment upper part 21 and the abutment lower part 23, in particular above the drive part 23a. This predetermined breaking point 45 can be designed in such a way that the abutment upper part 21 breaks off when forces harmful to the anchoring part 30 occur. This effectively prevents damage to the anchoring part 30. The abutment lower part 23, which is preferably made of plastic, and which remains in the anchoring part 30 after a corresponding fracture, can easily be removed.

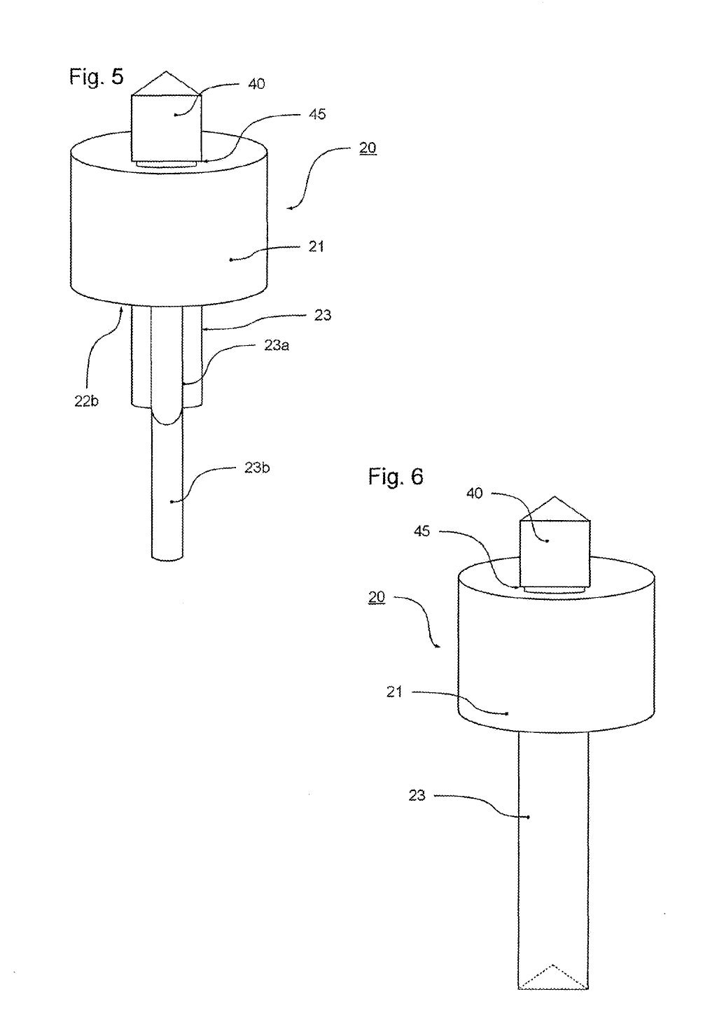

In another embodiment (cf. FIG. 5), the tool receiving portion 40 is not countersunk in the abutment upper part 21 but protrudes above it. FIG. 5 shows a triangle which protrudes above the abutment upper part cover area 22a and can be received by a correspondingly designed tool (e.g. a triangular wrench).

A notch between the tool receiving portion 40 from FIG. 5 and the abutment upper part 21 forms the predetermined breaking point 45 which can break off in a controlled manner. The embodiment according to FIG. 5 has the advantage that a fully functioning abutment 20 remains even after the tool receiving portion 40 has broken off. In this respect, it is possible to deliberately cause the tool receiving portion 40 to break off. Alternatively, after successfully introducing the anchoring part 30, the excess length in the form of the tool receiving portion 40 can be ground off. Finishing work on the abutment upper part 21 can be carried out if necessary. In this respect, it is possible to use the abutment 20 not only as an insertion aid but also as a component of the artificial dental prosthesis.

While in FIG. 5 the drive part 23a and the retention part 23b resemble the drive parts 23a and retention parts 23b of the embodiments according to FIGS. 3 and 4, FIG. 6 provides an abutment lower part 23 that has a triangular base area. The abutment lower part 23 according to FIG. 6 is therefore an elongated triangle which, similarly to the hexalobular shape from FIGS. 3 and 5, can be introduced into a corresponding receiving channel 36. Here too, the result is a form-fit which guarantees that the forces exerted on the abutment 20 are effectively transferred to the anchoring part 30.

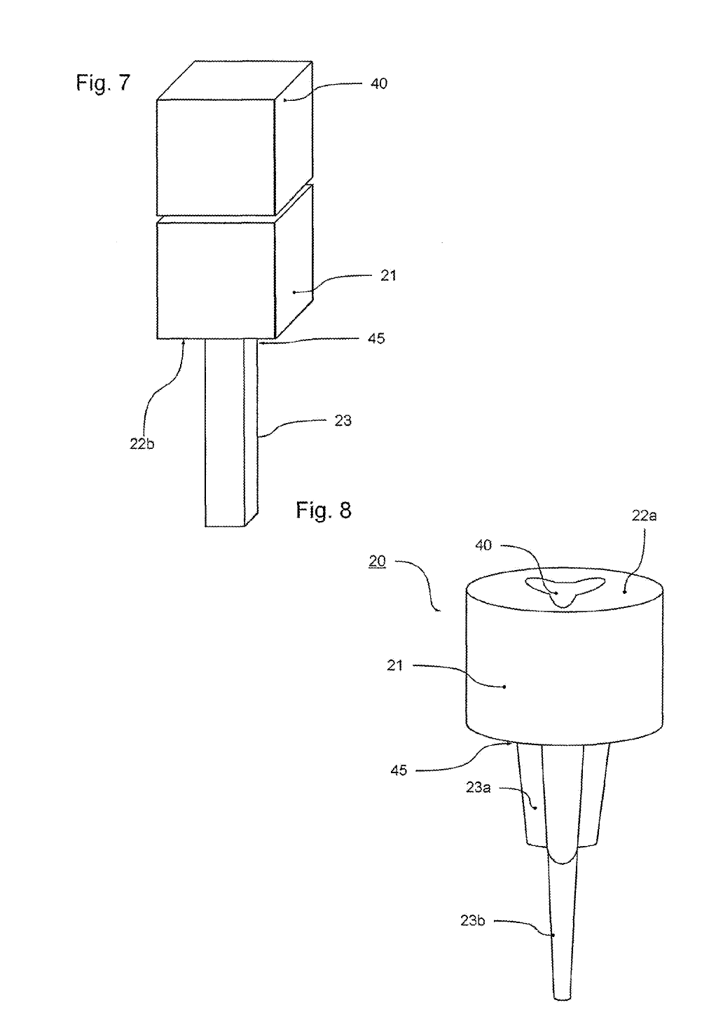

FIG. 7 shows a further embodiment of the abutment 20 according to the invention. The abutment upper part 21 and the abutment lower part 23 are designed in the shape of a cuboid. The bases of the cuboids are squares, the cuboid of the abutment lower part 23 resting centrally on the abutment upper part base area 22b. The abutment lower part 23 thus forms a square which can be introduced form-fittingly into a correspondingly designed receiving channel 36. The cuboid abutment upper part 21 can be ground, like the abutment upper parts 21 already explained. In the unground form, however, the whole abutment upper part 21 also forms a square which can be used as a tool receiving portion 40 for a correspondingly designed tool wrench. Ultimately, the wrench engages form-fittingly on at least two alternately arranged lateral surfaces of the cuboid of the abutment upper part 21 such that a torque can be applied. The abutment upper part 21 thus forms the tool receiving portion 40 which is used for applying suitable forces. Thus a correspondingly designed anchoring part 30 can be screwed into a bone 2 by the abutment 20 according to FIG. 7. The abutment 21 is preferably made up of two cubes, the cubes being joined together to form a predetermined breaking point 45. Thus, if the tool wrench is attached in the upper region of the abutment 21, it is possible to effectively limit the force transferred to the anchoring part 30.

FIG. 8 shows another embodiment of the abutment 20 according to the invention. Compared to the abutment from FIG. 4, in this case the abutment lower part 23 is designed conically overall, such that the diameter of the abutment lower part 23 decreases from the top down. Both the drive part 23a with the cloverleaf design and also the retention part 23b can taper towards the bottom.

FIG. 11 shows a further abutment 20 according to the invention which has no rotationally symmetrical design in respect of its longitudinal axis 7. Ultimately, the abutment upper part 21 is a finite cylinder with an elliptical abutment upper part cover area 22a and an elliptical abutment upper part base area 22b. A slotted hole is provided as tool receiving portion 40, the hole being arranged in the cylinder in such a way that the length of the slotted hole can be maximized, sufficient material remaining in the edge regions such that no damage occurs to the abutment 20 when inserting a tool. Abutment lower part 23 also has a correspondingly designed drive part 23a which is likewise designed as a cylinder with elliptical, preferably oval, base and cover areas. The retention part 23b can have a circular cross-section or also any other cross-section, in particular an elliptical cross-section.

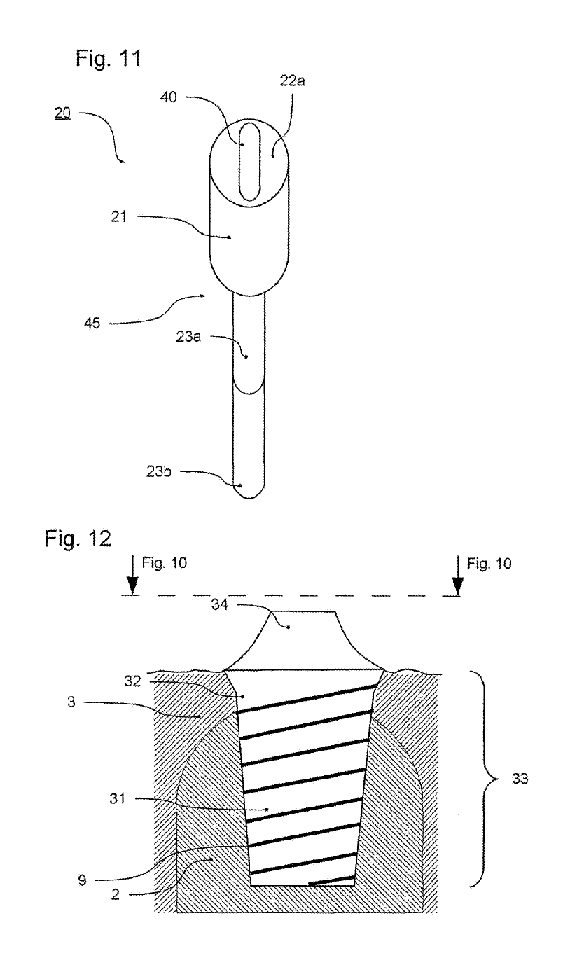

A corresponding abutment 20 can be used in an especially advantageous manner in conjunction with an elongated anchoring part 30, as is shown in FIGS. 10 and 12. A corresponding anchoring part 30 has a cylindrical lower subgingival section 33. In the upper subgingival section 33, the anchoring part 30 widens such that a truncated cone with an elliptical cover area emerges here. Accordingly, the shoulder section 34 and the receiving region 37 are also correspondingly elliptical. The receiving channel 36 can then be designed in the shape of a slotted hole that is particularly suitable for absorbing torsional forces. In this respect, it is easily possible to establish a transfer of forces between the abutment 20 from FIG. 11 and the anchoring part 30 from FIGS. 10 and 12.

Generally in the field of dentistry, the problem is that screw-in anchoring parts 30 can be of very small dimensions. For example, the lower subgingival section 33 can have a diameter of less than 5 mm, in particular less than 4 mm, in particular less than 3 mm. It proves to be extremely problematic to provide a receiving channel 36 on such small anchoring parts 30 which is suitable for absorbing sufficiently high forces, e.g. greater than 30 Nm. The present invention therefore proposes to provide the anchoring part 30 with an elongated upper region (e.g. an elongated shoulder section 34). Anchoring parts 30 designed in such a way may be particularly suitable for producing artificial dental prostheses for premolars. In addition, they are suitable for the attachment of slot-shaped openings, such as are shown, for example, in FIG. 10. The slot-shaped opening according to the invention, preferably in the form of a slotted hole, makes it easier to screw the anchoring parts 30 into the bone 2.

Based on the embodiments described, it should be clear that the abutment lower part 23 or a partial region thereof (e.g. the drive part 23a) can have very different shapes which achieve the aim according to the invention, namely a form-fit with a correspondingly designed receiving channel 36. Polygonal profiles, e.g. triangular, square, pentagonal, hexagonal, etc. or hexalobular shapes, are conceivable, such as are familiar from torx screw bits.

Moreover, there are numerous different possibilities for where the tool receiving portion 40 is provided on the abutment 20. As described, the tool receiving portion 40 can be countersunk in the abutment upper part 21 (e.g. FIG. 4) and/or mounted on it (e.g. FIG. 5). In addition, the abutment upper part 21 can have a shape which provides the functionality of a tool receiving portion 40.

Furthermore, the predetermined breaking point 45 can be provided in different positions as required. Thus it is conceivable, for example, not to provide the predetermined breaking point 45 for the abutment 20 from FIG. 4 between the abutment upper part 21 and the abutment lower part 23, but rather to provide an appropriate predetermined breaking point centrally, as shown for example in FIG. 7, on the abutment upper part 21. In this respect, it is possible to create an abutment 20 that can be inserted functionally, even after initiation of the predetermined break, for creating an artificial dental prosthesis. A large number of possible variations emerge with regard to the design of the abutment upper part 21. For example, the abutment upper part 21 from FIG. 4 can be dimensioned in such a way that, after the predetermined break or after removal of the tool receiving portion 40, an abutment upper part 21 remains as is shown in FIG. 3. Furthermore, the predetermined breaking point 45 can be provided in different positions as required, e.g. on the abutment upper part 21. For example, it can be located centrally, as shown in FIG. 7, or in the upper or lower third of the abutment upper part 21. It should be clear to the person skilled in the art working here that the position of the predetermined breaking point 45 is significantly responsible for the appearance of the abutment 20 after a predetermined break.

The abutment 20 according to the invention is described in the preceding embodiments in conjunction with a crown 1. The abutment described can support any superstructures in place of the crown 1.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-17. (canceled)

18. An abutment for insertion into a receiving region of an anchoring part of an artificial dental prosthesis, the abutment comprising:
an abutment upper part disposed along a longitudinal axis and
an abutment lower part having a profile for form-fitting insertion of the abutment into the receiving region of the anchoring part, such that a torque applied to the abutment is transferable to the anchoring part,
wherein the abutment upper part comprises a tool receiving portion for form-fittingly receiving a tool.

19. The abutment according to claim 18, wherein the tool receiving portion comprises an appendage which sits on the abutment upper part along the longitudinal axis.

20. The abutment according to claim 19, wherein the appendage sits on the abutment upper part on a side directed away from the abutment lower part.

21. The abutment according to claim 18, further comprising a predetermined breaking point which limits torque transmission between abutment upper part and abutment lower part and/or between tool receiving portion and abutment lower part.

22. The abutment according to claim 21, wherein the predetermined breaking point comprises at least one notch.

23. The abutment according to claim 18, wherein the abutment lower part is configured such that it can be inserted into the abutment receiving region of the anchoring part.

24. The abutment according to claim 18, wherein the profile of the abutment comprises a polygonal section and/or a hexalobular shape.

25. The abutment according to claim 18, wherein the tool receiving portion comprises a polygonal section and/or a hexalobular shape and/or a receiving portion for a polygonal section and/or a receiving portion for a hexalobular shape.

26. The abutment according to claim 18, wherein the abutment is grindable at least in sections and optionally comprises a plastic.

27. The abutment according to claim 26, wherein the plastic comprises fiberglass-reinforced and/or carbon fiber-reinforced plastic, wherein the fibers are aligned parallel to the longitudinal axis.

28. The abutment according to claim 18, wherein the abutment upper part is widened in relation to the abutment lower part and/or is configured as outwardly protruding to create a contact surface, wherein the contact surface extends essentially perpendicular to the longitudinal axis.

29. An artificial dental prosthesis having an implant for receiving a crown, wherein the implant comprises an abutment according to claim 18 and an anchoring part, wherein the anchoring part comprises an abutment receiving region for receiving the abutment and is formed at least in sections from a first material, the first material selected from the group of technical ceramics, optionally oxide ceramics.

30. The artificial dental prosthesis according to claim 29, wherein the anchoring part has a frustoconical section.

31. The artificial dental prosthesis according to claim 30, wherein the frustoconical section has a concave circumferential surface for receiving part of the crown.

32. The artificial dental prosthesis according to claim 29, wherein the anchoring part comprises at least one threaded section for screwing the anchoring part into a bone.

33. The artificial dental prosthesis according to claim 29, wherein the anchoring part is formed such that at least one cross-sectional area has an essentially oval, optionally elliptical, area boundary.

34. The artificial dental prosthesis according to claim 33, wherein the abutment receiving region comprises an elongated hole aligned along an axis of symmetry of the oval, optionally elliptical, area boundary of the anchoring part.

35. A method for screwing an anchoring part into biological tissue, the method comprising using the abutment according to claim 18 exclusively as a screwing aid.

36. A method for producing and/or implanting the artificial dental prosthesis according to claim 29, the method comprising steps of:
producing an individualized anchoring part having an abutment receiving region;
introducing a section of an abutment into the abutment receiving region for form-fitting joining of the abutment to the anchoring part;
attaching a tool to the abutment; and
applying a torque with the tool to screw the anchoring part into a bone.

37. The method according to claim 36, further comprising a step of removing sections of the abutment for adapting the abutment to patient-specific circumstances.
